(19) United States
(12) Patent Application Publication
(10) Pub. No.: US 2023/0355438 A1
(43) Pub. Date: Nov. 9, 2023

(54) INTRAOCULAR DRAINAGE TUBE WITH VALVE

(71) Applicant: New World Medical, Inc., Rancho Cucamonga, CA (US)

(72) Inventors: John KOONTZ, Chino, CA (US); Eric PORTEOUS, Corona, CA (US); Jason MOHR, Fontana, CA (US); Khalid MANSOUR, Eastvale, CA (US)

(21) Appl. No.: 18/028,440

(22) PCT Filed: Oct. 12, 2021
(86) PCT No.: PCT/US2021/054631

§ 371 Date: Mar. 24, 2023

Related U.S. Application Data
(60) Provisional application No. 63/091,695, filed on Oct. 14, 2020.

(51) Int. Cl. A61F 9/007

(52) U.S. Cl. CPC A61F 9/00781; A61F 9/00736; A61F 2250/0013

(57) ABSTRACT

Intraocular drainage devices are provided. An intraocular drainage device includes a drainage tube that provides for increased drainage of aqueous humour from the anterior chamber of the eye. A self-clearing device is disposed within the drainage tube. The self-clearing device provides an increased fluid flow path within the drainage tube in response to clotting from the body or clogging from debris in the aqueous humour. Methods of manufacturing intraocular drainage devices and methods of inserting intraocular drainage devices into the eye are also provided.

### CROSS-REFERENCES TO RELATED APPLICATIONS

This Application claims the benefit of U.S. Provisional Patent Application No. 63/091,695 entitled “INTRAOCULAR DRAINAGE TUBE WITH VALVE,” filed on Oct. 14, 2020, the disclosure of which is hereby incorporated by reference in its entirety for all purposes.

### TECHNICAL FIELD

The present disclosure generally relates to an intraocular drainage device, in particular an intraocular drainage tube having a valve to clear blockages.

### BACKGROUND

Aqueous humour typically drains from the anterior chamber of the eye via the trabecular meshwork at the edge of the cornea. In some circumstances, overproduction of aqueous humour or reduced drainage of aqueous humour can increase intraocular pressure which can cause discomfort and/or damage the optic nerve. Intraocular drainage devices are used to provide increased drainage of aqueous humour from the anterior chamber, particularly for glaucoma patients. However, some typical intraocular drainage devices have a tube with a very small diameter that is subject to clotting or clogging due to the body healing after insertion of the intraocular drainage device and/or debris in the aqueous humour. Some typical intraocular drainage devices have large diameter drainage tubes in an attempt to mitigate the clogging issue. Accordingly, it is desirable to provide an intraocular drainage device having a small diameter flow path or drainage tube that can clear clots or debris to restore desirable fluid flow without requiring surgical intervention.

### SUMMARY

In one or more embodiments, an intraocular drainage device includes a drainage tube comprising an interior surface defining a fluid flow path and a self-clearing device disposed within the drainage tube and across the fluid flow path, the self-clearing device including a flow path opening configured to provide a selected fluid flow pressure when the self-clearing device is in an unstressed state.

It is to be understood that both the foregoing general description and the following detailed description are exemplary and explanatory and are intended to provide further explanation of the disclosure as claimed.

### BRIEF DESCRIPTION OF THE DRAWINGS

The accompanying drawings, which are included to provide further understanding of the disclosure and are incorporated in and constitute a part of this specification, illustrate embodiments of the disclosure and together with the description serve to explain the principles of the disclosure.

FIG. 1 depicts a perspective view of an example intraocular drainage device, according to aspects of the disclosure.

FIG. 2 depicts another perspective view of the intraocular drainage device of FIG. 1, according to aspects of the disclosure.

FIG. 3 depicts a front view of the intraocular drainage device of FIG. 1, according to aspects of the disclosure.

FIG. 4 depicts a front view of an example intraocular drainage device, according to aspects of the disclosure.

FIG. 5 depicts a front view of an example intraocular drainage device, according to aspects of the disclosure.

FIG. 6 depicts a perspective view of an example intraocular drainage device, according to aspects of the disclosure.

FIG. 7 depicts a perspective view of an example intraocular drainage device, according to aspects of the disclosure.

FIG. 8 depicts a perspective view of an example intraocular drainage device, according to aspects of the disclosure.

FIG. 9 depicts a side view of the intraocular drainage device of FIG. 8, according to aspects of the disclosure.

FIG. 10 depicts a front view of the intraocular drainage device of FIG. 8, according to aspects of the disclosure.

FIG. 11 depicts a cross-sectional side view of an example intraocular drainage device, according to aspects of the disclosure.

FIG. 12 depicts a cross-sectional side view of an example intraocular drainage device, according to aspects of the disclosure.

FIG. 13 depicts a front view of the intraocular drainage device of FIG. 12, according to aspects of the disclosure.

### DETAILED DESCRIPTION

The detailed description set forth below describes various configurations of the subject technology and is not intended to represent the only configurations in which the subject technology may be practiced. The detailed description includes specific details for the purpose of providing a thorough understanding of the subject technology. Accordingly, dimensions are provided in regard to certain aspects as non-limiting examples. However, it will be apparent to those skilled in the art that the subject technology may be practiced without these specific details. In some instances, well-known structures and components are shown in block diagram form in order to avoid obscuring the concepts of the subject technology.

It is to be understood that the present disclosure includes examples of the subject technology and does not limit the scope of the appended claims. Various aspects of the subject technology will now be disclosed according to particular but non-limiting examples. Various embodiments described in the present disclosure may be carried out in different ways and variations, and in accordance with a desired application or implementation.

Normal eye pressure ranges from 12-22 mm Hg for people without glaucoma. As discussed above, for patients with glaucoma overproduction of aqueous humour or reduced drainage of aqueous humour can increase intraocular pressure (TOP) which can cause discomfort and/or damage the optic nerve. Thus, for glaucoma patients a typical biological aqueous humour flow rate may have an ideal pressure drop in a range around 12 mm of mercury. If the IOP is too low (e.g., less than 6 mm of mercury), there is a danger of becoming hypotonous where the eye structure essentially falls apart and may result in blindness in that eye. On the other hand, if the IOP is consistently too high (e.g., more than 14 mm of mercury) or has spikes above 18 mm of mercury, there is also a danger of deterioration and blindness in the eye. Accordingly, an IOP in the range of 8-14 mm of mercury is desirable to maintain optimal eye health.

For eye diseases such as Glaucoma, for example, underdrainage of the aqueous humour increases the pressure into the undesirable high pressure ranges discussed above. An intraocular drainage device (e.g., drainage tube) may be used to relieve this pressure by providing a tube that improves aqueous humour flow. Thus, it is very desirable that the drainage tube maintain the desired aqueous humour flow. However, if the drainage tube becomes partially or wholly blocked due to clotting or debris, the aqueous humour flow within the eye is reduced, thereby increasing the pressure. To overcome the clotting and debris clogging issues with drainage tubes, the present disclosure provides for intraocular drainage devices having a self-clearing device (e.g., relief feature, deformation element). The self-clearing device responds to a build-up in pressure due to clotting or debris by providing an increased fluid pathway (e.g., increased inner diameter, increased flow pattern, valve), which in turn provides increased fluid flow through the drainage tube, thus restoring the aqueous humour pressure to the desired pressure range.

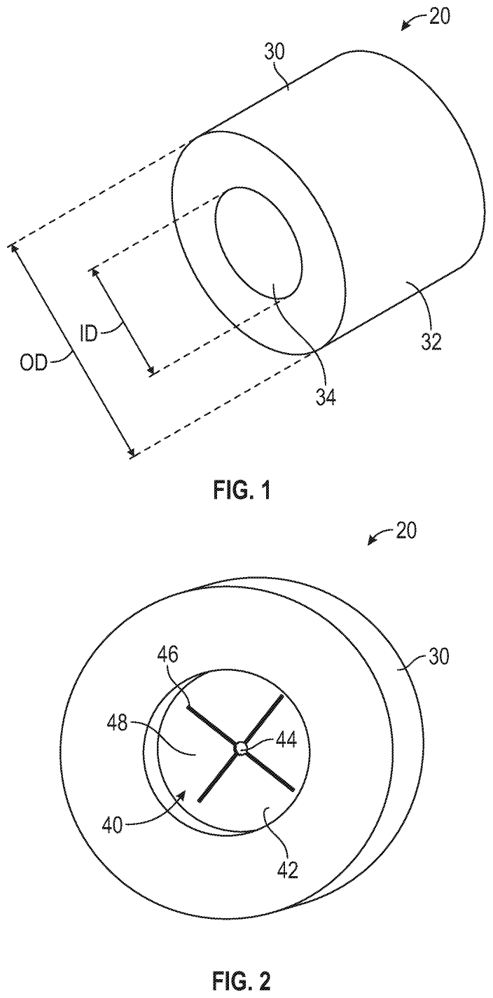

Referring now in more detail to the drawings in which like reference numerals refer to like or corresponding elements among the several views, there is shown in FIG. 1 an intraocular drainage device 20 having a drainage tube 30. The drainage tube 30 has an outer surface 32 having an outer diameter OD and an inner surface 34 having an inner diameter ID.

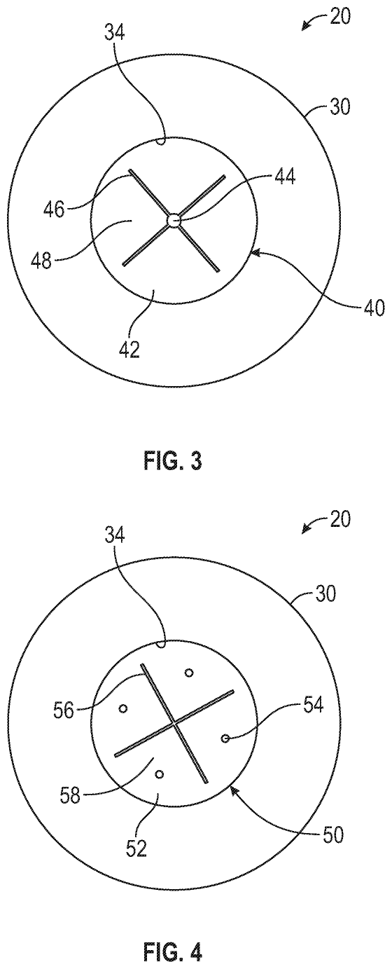

According to aspects of the disclosure, as shown in FIGS. 2 and 3, the intraocular drainage device 20 includes a self-clearing device 40 disposed within the inner diameter ID of the drainage tube 30. The self-clearing device 40 includes a membrane 42 disposed across the inner diameter ID. The membrane 42 may be formed integrally with the drainage tube 30 or it may be a separate component that is inserted into the drainage tube 30. The membrane 42 includes a flow path hole 44, slits 46 and flaps 48 defined by the slits 46. In some aspects, the membrane 42 may have a consistent thickness (e.g., 1 mm) across the inner diameter ID. In some aspects, the membrane 42 may have a radial thickness where the membrane 42 is thinner (e.g., 1 mm) near the flow path hole 44 and thicker (e.g., 2 mm) near the inner surface 34 of the drainage tube 30.

The flow path hole 44 may be sized and/or shaped so that the aqueous humour flow through the flow path hole 44 is within a desired pressure range (e.g., 8-14 mm of mercury) under free flowing conditions. If the inner diameter ID and/or the flow path hole 44 become partially or fully obstructed due to clotting or debris, fluid pressure will build up on the membrane 42. The increased fluid pressure on the membrane 42 causes at least one of the flaps 48 to open or move in a downstream direction of the fluid flow, thus increasing the fluid flow path beyond or greater than the flow path hole 44. The increased fluid flow path allows a greater flow of aqueous humour through the drainage tube 30, as well as a larger opening for clots or debris to fit through, thus clearing the obstruction through the drainage tube 30. After clearing the obstruction, the fluid pressure can return back to the initial fluid pressure level and the flap(s) 48 return to the initial orientation/position, thus returning to the desired fluid flow rate through the flow path hole 44.

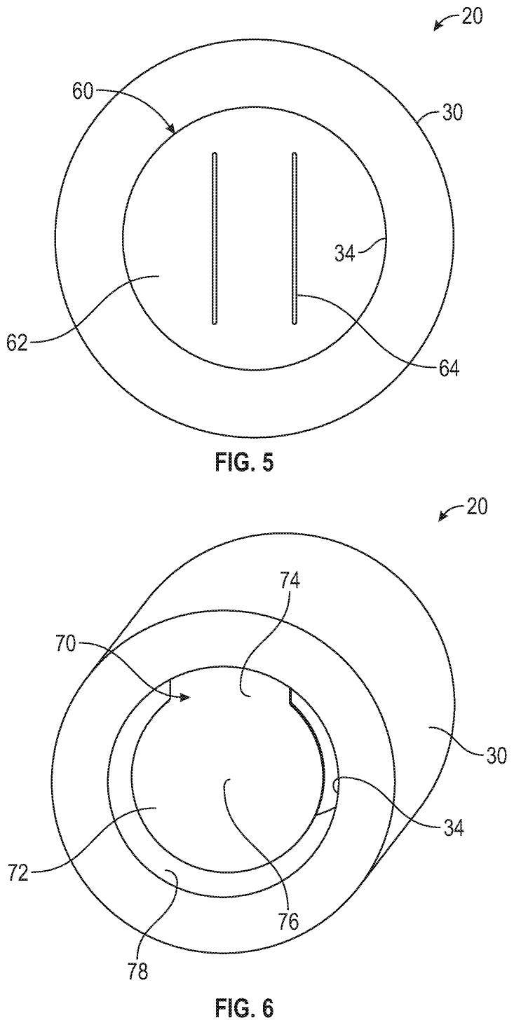

According to aspects of the disclosure, as shown in FIG. 4, the intraocular drainage device 20 includes a self-clearing device 50 disposed within the inner diameter ID of the drainage tube 30. The self-clearing device 50 includes a membrane 52 disposed across the inner diameter ID. The membrane 52 may be formed integrally with the drainage tube 30 or it may be a separate component that is inserted into the drainage tube 30. The membrane 52 includes multiple flow path holes 54, slits 56 and flaps 58 defined by the slits 56. In some aspects, the membrane 52 may have a consistent thickness (e.g., 1 mm) across the inner diameter ID. In some aspects, the membrane 52 may have a radial thickness where the membrane 52 is thinner (e.g., 1 mm) near the flow path hole 54 and thicker (e.g., 2 mm) near the inner surface 34 of the drainage tube 30.

The flow path holes 54 may be sized and/or shaped so that the total aqueous humour flow through the combination of flow path holes 54 is within a desired pressure range (e.g., 8-14 mm of mercury) under free flowing conditions. If the inner diameter ID and/or one or more of the flow path holes 54 become partially or fully obstructed due to clotting or debris, fluid pressure will build up on the membrane 52. The increased fluid pressure on the membrane 52 causes at least one of the flaps 58 to open or move in a downstream direction of the fluid flow, thus increasing the fluid flow path beyond or greater than provided by the flow path holes 54 themselves. The increased fluid flow path allows a greater flow of aqueous humour through the drainage tube 30, as well as a larger opening for clots or debris to fit through, thus clearing the obstruction through the drainage tube 30. After clearing the obstruction, the fluid pressure can return back to the initial fluid pressure level and the flap(s) 58 return to the initial orientation/position, thus returning to the desired fluid flow rate through the flow path holes 54.

According to aspects of the disclosure, as shown in FIG. 5, the intraocular drainage device 20 includes a self-clearing device 60 disposed within the inner diameter ID of the drainage tube 30. The self-clearing device 60 includes a membrane 62 disposed across the inner diameter ID. The membrane 62 may be formed integrally with the drainage tube 30 or it may be a separate component that is inserted into the drainage tube 30. The membrane 62 includes one or more slits 64. In some aspects, the membrane 62 may have a consistent thickness (e.g., 1 mm) across the inner diameter ID. In some aspects, the membrane 62 may have a radial thickness where the membrane 62 is thinner (e.g., 1 mm) towards the center of the membrane 62 and thicker (e.g., 2 mm) near the inner surface 34 of the drainage tube 30.

The slits 64 may be sized and/or shaped so that the total aqueous humour flow through the combination of slits 64 is within a desired pressure range (e.g., 8-14 mm of mercury) under free flowing conditions. If the inner diameter ID and/or one or more of the slits 64 become partially or fully obstructed due to clotting or debris, fluid pressure will build up on the membrane 62. The increased fluid pressure on the membrane 62 causes the membrane 62 to bulge or move in a downstream direction of the fluid flow, thus causing the slits 64 to widen to increase the fluid flow path beyond or greater than provided by the slits 64 themselves. The increased fluid flow path allows a greater flow of aqueous humour through the drainage tube 30, as well as a larger opening for clots or debris to fit through, thus clearing the obstruction through the drainage tube 30. After clearing the obstruction, the fluid pressure can return back to the initial fluid pressure level and the slits 64 return to the initial orientation/position, thus returning to the desired fluid flow rate through the slits 64.

According to aspects of the disclosure, as shown in FIG. 6, the intraocular drainage device 20 includes a self-clearing device 70 disposed within the inner diameter ID of the drainage tube 30. The self-clearing device 70 includes a membrane 72 disposed across the inner diameter ID. The membrane 72 may be formed integrally with the drainage tube 30 or it may be a separate component that is inserted into the drainage tube 30. A coupling portion 74 of the membrane 72 may be coupled to the inner surface 34 of the drainage tube 30, while the remaining portion of the membrane 72 is configured as a flap 76. The flap 76 has a diameter smaller than the inner diameter ID of the drainage tube 30, thus forming a crescent shaped flow path 78 disposed between the outer perimeter of the flap 76 and the inner diameter 34. In some aspects, the membrane 72 may have a consistent thickness (e.g., 1 mm) across the inner diameter ID. In some aspects, the membrane 72 may have a radial thickness where the membrane 72 is thinner (e.g., 1 mm) for the flap 76 and thicker (e.g., 2 mm) for the coupling portion 74.

The flap 76 may be sized and/or shaped so that the total aqueous humour flow through the crescent shaped flow path 78 is within a desired pressure range (e.g., 8-14 mm of mercury) under free flowing conditions. If the inner diameter ID and/or the crescent shaped flow path 78 become partially or fully obstructed due to clotting or debris, fluid pressure will build up on the membrane 72. The increased fluid pressure on the membrane 72 causes the flap 76 to move in a downstream direction of the fluid flow, thus causing the crescent shaped flow path 78 to widen to increase the fluid flow path. The increased fluid flow path allows a greater flow of aqueous humour through the drainage tube 30, as well as a larger opening for clots or debris to fit through, thus clearing the obstruction through the drainage tube 30. After clearing the obstruction, the fluid pressure can return back to the initial fluid pressure level and the flap 76 return to the initial orientation/position, thus returning to the desired fluid flow rate through the self-clearing device 70.

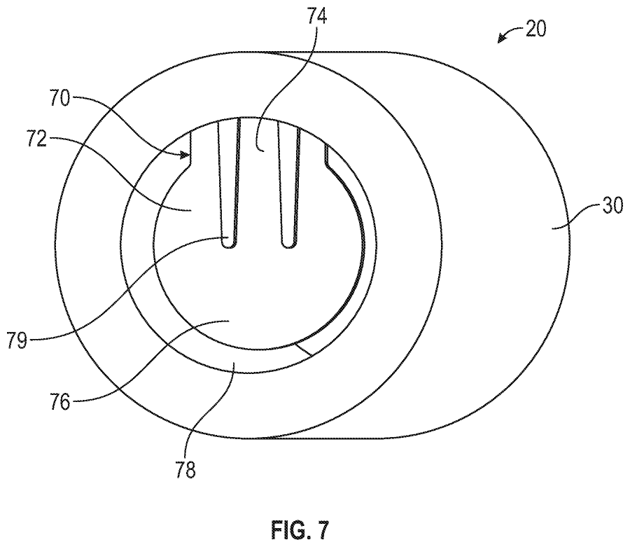

According to aspects of the disclosure, as shown in FIG. 7, the self-clearing device 70 may include structural members 79 (e.g., teeth) coupled to or integrally formed with the membrane 72. The structural members 79 may be sized and shaped to provide a desired stiffness to the flap 76. For example, the flap 76 may have a thickness of 1 mm and the structural members 79 may have a thickness of 2-3 mm. Accordingly, the flap 76 with the structural members 79 may require a higher fluid flow pressure to move than the flap 76 without the structural members 79 shown in FIG. 6.

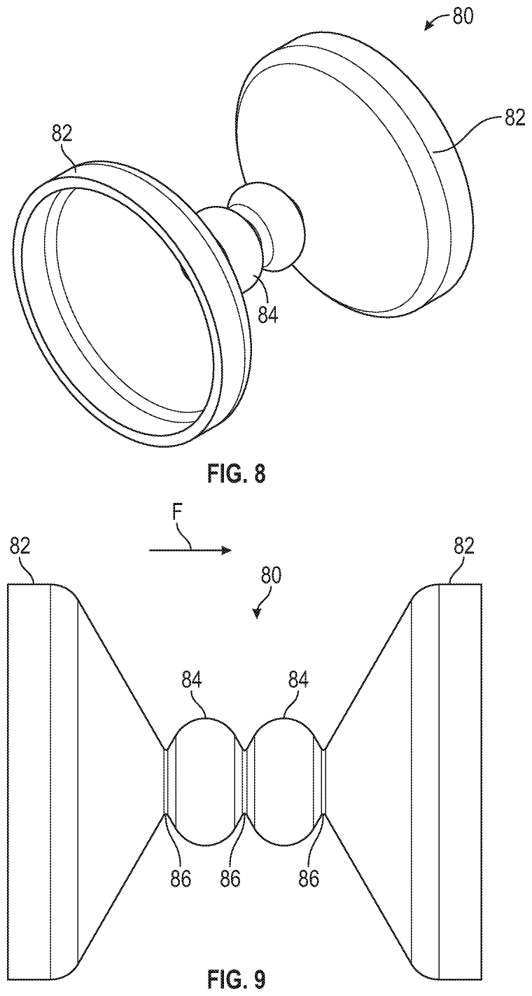

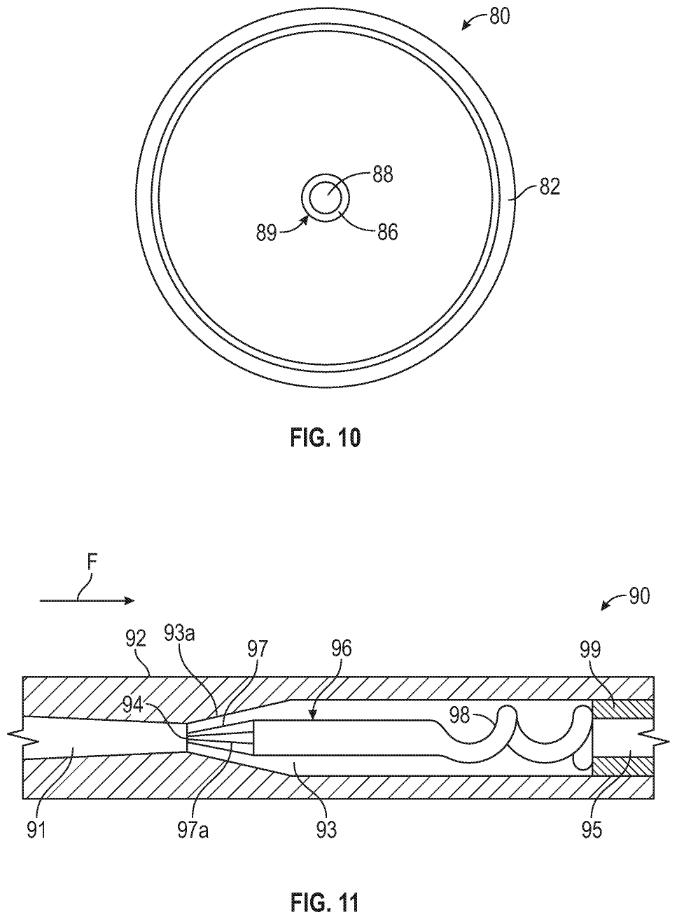

According to aspects of the disclosure, as shown in FIGS. 8-10, an intraocular drainage device 80 includes opposing funnel shaped end portions 82 connected by bulbous portions 84, providing the intraocular drainage device 80 with a dumbbell shape. Flexible connection portions 86 are disposed between the funnel shaped end portions 82 and the bulbous portions 84. A flow path hole 88 is disposed within each of the flexible connection portions 86 where the combination of flow path holes 88 and flexible connection portions 86 form a self-clearing device 89. In operation, the aqueous humour fluid flows from left to right in the intraocular drainage device 80 of FIG. 9, as indicated by flow arrow F.

The flow path holes 88 may be sized and/or shaped so that the total aqueous humour flow through the combination of flow path holes 88 is within a desired pressure range (e.g., 8-14 mm of mercury) under free flowing conditions. If one or more of the flow path holes 88 become partially or fully obstructed due to clotting or debris, fluid pressure will build up on one or more of the flexible connection portions 86. The increased fluid pressure causes the flexible connection portion(s) 86 to bulge out or move in a radial direction from the fluid flow, thus increasing the diameter of the fluid flow path hole(s) 88 and increasing the fluid flow path. The increased fluid flow path allows a greater flow of aqueous humour through the intraocular drainage device 80, as well as a larger opening for clots or debris to fit through, thus clearing the obstruction through the intraocular drainage device 80. After clearing the obstruction, the fluid pressure can return back to the initial fluid pressure level and the fluid flow path hole(s) 88 return to their initial diameter, thus returning to the desired fluid flow rate through the intraocular drainage device 80.

According to aspects of the disclosure, the intraocular drainage device 80 may have only one bulbous portion 84 or more than two bulbous portions 84, with a commensurate decrease or increase in the number of flexible connection portions 86. According to aspects of the disclosure, the intraocular drainage device 80 may have no bulbous portions 84, so that the opposing funnel shaped end portions 82 are connected by a single flexible connection portion 86, resulting in an hourglass shape. According to aspects of the disclosure, the intraocular drainage device 80, the inner diameter of the widest portion of the funnel shaped end portion 82 may be about 300 microns and the inner diameter of the flow path hole 88 in an unstressed state (e.g., resting position, not expanded) may be about 30 microns.

According to aspects of the disclosure, as shown in FIG. 11, an intraocular drainage device 90 includes a drainage tube 92 having a flow path hole 94. An upstream flow path 91 is disposed on the upstream side of the flow path hole 94 and a downstream flow path 93 is disposed on the downstream side of the flow path hole 94. Each of the upstream and downstream flow paths 91, 93 may have a frustoconical shape that narrows down at the flow path hole 94. A valve 96 is disposed on a downstream side of the fluid flow hole 94. The valve 96 may have a conical portion 97 with the narrow portion of the conical portion 97 disposed next to the fluid flow hole 94. The valve 96 also includes a spring 98 coupled to the conical portion 97 on one end and coupled to or engaged with a stop member 99 on the other end. The stop member 99 includes an exit flow path 95.

In operation, the aqueous humour fluid flows from left to right in the intraocular drainage device 90 of FIG. 11, as indicated by arrow F. In an unstressed state of the spring 98 (e.g., spring 98 fully extended), the conical portion 97 may fully or partially block the fluid flow hole 94, thus preventing or limiting fluid flow. As fluid pressure builds up on the conical portion 97, the conical portion 97 is forced to move in the flow direction F and the spring 98 is compressed. In this stressed (e.g., compressed) state of the spring 98, fluid flows around the conical portion 97, through an interior channel of the spring 98 and out the exit flow path 95.

According to aspects of the disclosure, different fluid flow rates may be provided by providing axial grooves 97a on the conical portion 97 of the valve 96 and/or axial grooves 93a on the interior surface of the drainage tube 92 along at least a portion of the downstream flow path 93. Such grooves provide an increased flow path area in the vicinity of the flow path hole 94, thus allowing at least minimal fluid flow in the unstressed state of the spring 98 (e.g., increased fluid flow without moving the spring 98). According to aspects of the disclosure, the intraocular drainage device 90 may also include an O-ring. For example, the conical portion 97 may have an O-ring embedded on the cone. According to aspects of the disclosure, the conical portion 97 may be formed of a soft material and the spring may be formed of a metal such as NiTi.

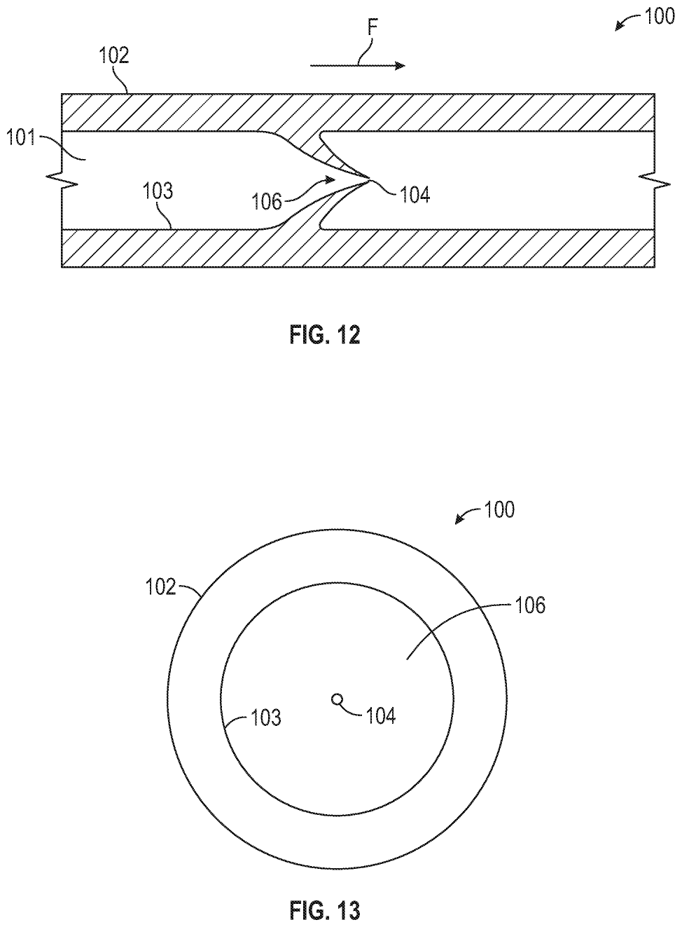

According to aspects of the disclosure, as shown in FIGS. 12 and 13, an intraocular drainage device 100 includes a drainage tube 102 having an internal flow path 101 and a valve 106 disposed across the internal flow path 101. The valve 106 includes a flow path hole 104 and the fluid flow is in the direction indicated by the flow arrow F. The valve 106 may be a vein style valve having a thickness of about 2 mm adjacent to an internal surface 103 of the drainage tube 102 and tapering down to a thickness of about 1 mm near the flow path hole 104. According to aspects of the disclosure, the valve 106 may be any desirable valve that allows for a desired flow within a desired pressure range (e.g., 8-14 mm of mercury) in an unstressed state and that has a cracking pressure for which the valve will open up to provide an increased fluid flow to clear clots or debris. For example, the valve 106 may be a duckbill valve or a check valve, according to aspects of the disclosure.

According to aspects of the disclosure, desirable standard fluid flow pressure (e.g., 12 mm of pressure drop) may be provided by sizing the length and diameter of a restriction portion of an intraocular drainage device. For example, the intraocular drainage device may have an hourglass shape (e.g., intraocular drainage device 80) having a total length of about 10 mm, where a restrictive portion (e.g., center portion) of the tube provides the majority (e.g., 99% or greater) of the resistance/pressure drop of the intraocular drainage device. Here, the length of the intraocular drainage device on either side of the resistive central portion does not affect the resistance/pressure drop. Thus, according to aspects of the disclosure, the resistance to the fluid flow may come from the single restrictive portion of the intraocular drainage device, where the length and diameter of the restrictive portion may be varied to achieve the desired pressure drop of 12 mm of mercury, as shown in the following examples.
For example if the restriction is ˜1 mm in length the diameter would be ˜30 microns
For example if the restriction is ˜2 mm in length the diameter would be ˜35 microns
For example if the restriction is ˜3 mm in length the diameter would be ˜39 microns
For example if the restriction is ˜4 mm in length the diameter would be ˜42 microns
For example if the restriction is ˜5 mm in length the diameter would be ˜44 microns
For example if the restriction is ˜0.8 mm in length the diameter would be ˜28 microns
For example if the restriction is ˜0.6 mm in length the diameter would be ˜26 microns
For example if the restriction is ˜0.4 mm in length the diameter would be ˜24 microns
For example if the restriction is ˜0.2 mm in length the diameter would be ˜20 microns
For example if the restriction is ˜0.1 mm in length the diameter would be ˜17 microns
   According to aspects of the disclosure, the shorter the length of the restriction (e.g., restrictive portion), the smaller the diameter of the restriction as well. According to aspects of the disclosure, a shorter restriction length may make construction flexibility, expandability and blockage relief of the intraocular drainage device easier.

According to aspects of the disclosure, any of the above described intraocular drainage devices may be manufactured as a single integral device or as separate components that are assembled together. For example, a single integral device may be manufactured by a molding process in which the intraocular drainage device is molded as a solid body while using pins to create the interior spaces or flow path holes (e.g., two core pins, one from each end, that mate in the middle. As another example, the body of the intraocular drainage device or the drainage tube may be formed as one component and the self-clearing device may be formed as a separate component, for which the self-clearing device may be formed of a firmer material and/or process in order to be inserted or pushed into the body/drainage tube without deformation of the self-clearing device. According to aspects of the disclosure, any components of the above described intraocular drainage devices may be manufactured from materials including silicon (e.g., soft tube), plastic (e.g., self-clearing device for insertion into soft tube), hydrophobic polyurethane, hydrophobic acrylic, shape memory plastic and the like.

According to aspects of the disclosure, any of the above described intraocular drainage devices may be surgically implanted in the eye by either an ab externo process or an ab interno process. An ab externo process may include dissecting the outside conjuctiva of the eye, inserting (e.g., poking in) the drainage tube into the anterior chamber, suturing the drainage tube in place, and reapproximating and suturing the conjunctiva closed. Here, an end of the drainage tube is the only element that enters the interior of the eye (e.g., the anterior chamber). On the other hand, an ab interno process may include placing the intraocular drainage device into a medical instrument (e.g., an inserter), inserting the inserter into the eye and poking the drainage tube through into the interior of the eye (e.g., the anterior chamber) or depositing the drainage tube within the interior of the eye. The outflow end of the drainage tube may be placed near the side of the eye next to the nose of the patient. A surgical procedure for inserting an intraocular drainage device having a plate may include dissecting the external surface of the eye, inserting the plate into the external surface, insert the drainage tube into the interior of the eye, tie off the drainage tube, wait 2-3 weeks for the external surface of the eye to heal around the plate to trap the plate in the external eye material, untying the drainage tube so that back pressure starts a fluid flow.

In one or more embodiments, an intraocular drainage device comprises a drainage tube comprising an interior surface defining a fluid flow path; and a self-clearing device disposed within the drainage tube and across the fluid flow path, the self-clearing device comprising a flow path opening configured to provide a selected fluid flow pressure when the self-clearing device is in an unstressed state.

In aspects of the disclosure, the self-clearing device is a membrane. In aspects of the disclosure, the membrane comprises: a flow path hole configured as the flow path opening, the flow path hole disposed in the center of the membrane; and a plurality of flaps, each flap defined by two slits in the membrane that extend radially from the flow path hole towards the interior surface of the drainage tube. In aspects of the disclosure, the membrane is configured such that at least a portion of one flap moves in a downstream direction of the fluid flow path in response to an increased fluid pressure on the membrane, thereby increasing the size of the flow path opening. In aspects of the disclosure, the membrane comprises: a plurality of flaps, each flap defined by two slits in the membrane that extend radially from the center of the membrane towards the interior surface of the drainage tube; and a flow path hole disposed in one or more of the flaps, wherein a combination of the flow path holes is configured as the flow path opening in the unstressed state.

In aspects of the disclosure, the membrane is configured such that at least a portion of one flap moves in a downstream direction of the fluid flow path in response to an increased fluid pressure on the membrane, thereby increasing the size of the flow path opening. In aspects of the disclosure, the membrane comprises: one or more slits disposed in the membrane, each slit having an open area that fluid can flow through, wherein a combination of the open area of the slits is configured as the flow path opening in the unstressed state. In aspects of the disclosure, the membrane is configured such that at least a portion of membrane bulges in a downstream direction of the fluid flow path in response to an increased fluid pressure on the membrane, thereby increasing the size of the open area of at least one slit and thus increasing the flow path opening. In aspects of the disclosure, the membrane comprises: a coupling portion coupled to the interior surface of the drainage tube; and a flap connected to the coupling portion, wherein an outer perimeter of the flap and the interior surface of the drainage tube define the flow path opening as a crescent shaped area.

In aspects of the disclosure, the membrane is configured such that at least a portion of the flap moves in a downstream direction of the fluid flow path in response to an increased fluid pressure on the membrane, thereby increasing the size of the crescent shaped area and thus increasing the flow path opening. In aspects of the disclosure, the membrane further comprises at least one structural member disposed across portions of the coupling portion and the flap, the at least one structural member configured to provide an increased resistance of the flap to movement. In aspects of the disclosure, the drainage tube comprises two opposing funnel shaped end portions, and wherein the self-clearing device comprises a flexible connecting portion having a flow path hole disposed through the center of the flexible connecting portion. In aspects of the disclosure, the flexible connecting portion is configured to bulge outward in a radial direction in response to an increased fluid pressure on the flexible connecting portion, thereby increasing the size of the flow path hole.

In aspects of the disclosure, the self-clearing device comprises: one or more bulbous portions disposed between the opposing funnel shaped end portions, wherein a flexible connecting portion is disposed on each side of each of the one or more bulbous portions. In aspects of the disclosure, the flexible connecting portion is a restriction portion providing 99 percent or greater of a fluid pressure drop of the intraocular drainage device, and wherein a diameter of the restriction portion may be sized based on a length of the restriction portion to achieve a predetermined fluid pressure drop. In aspects of the disclosure, the drainage tube comprises: an upstream flow path; a downstream flow path; a flow path hole defined by a junction of the upstream flow path and the downstream flow path; a stop member disposed on a downstream end of the drainage tube; and an exit flow path disposed through the stop member. In aspects of the disclosure, the self-clearing device is a valve disposed within the downstream flow path, the valve comprising: a conical portion disposed adjacent the flow path hole; and a spring, wherein one end of the spring is connected to the conical portion and the other end of the spring engages with the stop member.

In aspects of the disclosure, the conical portion of the valve is configured to move downstream in response to an increased fluid pressure on the conical portion, thereby compressing the spring. In aspects of the disclosure, the valve is configured to allow an aqueous humour fluid to flow through the flow path hole, around an exterior surface of the conical portion, through a center portion of the spring and out the exit flow path. In aspects of the disclosure, the self-clearing device is a valve, the valve comprising: a valve membrane; and a flow path hole disposed in the center of the valve membrane, wherein the valve membrane is configured to bulge downstream in response to an increased fluid pressure on valve membrane, thereby increasing the size of the flow path hole.

It is understood that any specific order or hierarchy of blocks in the methods of processes disclosed is an illustration of example approaches. Based upon design or implementation preferences, it is understood that the specific order or hierarchy of blocks in the processes may be rearranged, or that all illustrated blocks be performed. In some implementations, any of the blocks may be performed simultaneously.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

As used herein, the phrase “at least one of” preceding a series of items, with the term “or” to separate any of the items, modifies the list as a whole, rather than each item of the list. The phrase “at least one of” does not require selection of at least one item; rather, the phrase allows a meaning that includes at least one of any one of the items, and/or at least one of any combination of the items, and/or at least one of each of the items. By way of example, the phrase “at least one of A, B, or C” may refer to: only A, only B, or only C; or any combination of A, B, and C.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

It is understood that the specific order or hierarchy of steps, operations or processes disclosed is an illustration of exemplary approaches. Based upon design preferences, it is understood that the specific order or hierarchy of steps, operations or processes may be rearranged. Some of the steps, operations or processes may be performed simultaneously. Some or all of the steps, operations, or processes may be performed automatically, without the intervention of a user. The accompanying method claims, if any, present elements of the various steps, operations or processes in a sample order, and are not meant to be limited to the specific order or hierarchy presented.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein, but are to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intraocular drainage device, comprising:
a drainage tube comprising an interior surface defining a fluid flow path; and
a self-clearing device disposed within the drainage tube and across the fluid flow path, the self-clearing device comprising a flow path opening configured to provide a selected fluid flow pressure when the self-clearing device is in an unstressed state.

2. The intraocular drainage device of claim 1, wherein the self-clearing device is a membrane.

3. The intraocular drainage device of claim 2, wherein the membrane comprises:
a flow path hole configured as the flow path opening, the flow path hole disposed in the center of the membrane; and
a plurality of flaps, each flap defined by two slits in the membrane that extend radially from the flow path hole towards the interior surface of the drainage tube.

4. The intraocular drainage device of claim 3, wherein the membrane is configured such that at least a portion of one flap moves in a downstream direction of the fluid flow path in response to an increased fluid pressure on the membrane, thereby increasing the size of the flow path opening.

5. The intraocular drainage device of claim 2, wherein the membrane comprises:
a plurality of flaps, each flap defined by two slits in the membrane that extend radially from the center of the membrane towards the interior surface of the drainage tube; and
a flow path hole disposed in one or more of the flaps, wherein a combination of the flow path holes is configured as the flow path opening in the unstressed state.

6. The intraocular drainage device of claim 5, wherein the membrane is configured such that at least a portion of one flap moves in a downstream direction of the fluid flow path in response to an increased fluid pressure on the membrane, thereby increasing the size of the flow path opening.

7. The intraocular drainage device of claim 2, wherein the membrane comprises:
one or more slits disposed in the membrane, each slit having an open area that fluid can flow through, wherein a combination of the open area of the slits is configured as the flow path opening in the unstressed state.

8. The intraocular drainage device of claim 7, wherein the membrane is configured such that at least a portion of membrane bulges in a downstream direction of the fluid flow path in response to an increased fluid pressure on the membrane, thereby increasing the size of the open area of at least one slit and thus increasing the flow path opening.

9. The intraocular drainage device of claim 2, wherein the membrane comprises:
a coupling portion coupled to the interior surface of the drainage tube; and
a flap connected to the coupling portion, wherein an outer perimeter of the flap and the interior surface of the drainage tube define the flow path opening as a crescent shaped area.

10. The intraocular drainage device of claim 9, wherein the membrane is configured such that at least a portion of the flap moves in a downstream direction of the fluid flow path in response to an increased fluid pressure on the membrane, thereby increasing the size of the crescent shaped area and thus increasing the flow path opening.

11. The intraocular drainage device of claim 9, wherein the membrane further comprises at least one structural member disposed across portions of the coupling portion and the flap, the at least one structural member configured to provide an increased resistance of the flap to movement.

12. The intraocular drainage device of claim 1, wherein the drainage tube comprises two opposing funnel shaped end portions, and wherein the self-clearing device comprises a flexible connecting portion having a flow path hole disposed through the center of the flexible connecting portion.

13. The intraocular drainage device of claim 12, wherein the flexible connecting portion is configured to bulge outward in a radial direction in response to an increased fluid pressure on the flexible connecting portion, thereby increasing the size of the flow path hole.

14. The intraocular drainage device of claim 12, wherein the self-clearing device comprises:
one or more bulbous portions disposed between the opposing funnel shaped end portions, wherein a flexible connecting portion is disposed on each side of each of the one or more bulbous portions.

15. The intraocular drainage device of claim 12, wherein the flexible connecting portion is a restriction portion providing 99 percent or greater of a fluid pressure drop of the intraocular drainage device, and wherein a diameter of the restriction portion may be sized based on a length of the restriction portion to achieve a predetermined fluid pressure drop.

16. The intraocular drainage device of claim 1, wherein the drainage tube comprises:
an upstream flow path;
a downstream flow path;
a flow path hole defined by a junction of the upstream flow path and the downstream flow path;
a stop member disposed on a downstream end of the drainage tube; and
an exit flow path disposed through the stop member.

17. The intraocular drainage device of claim 16, wherein the self-clearing device is a valve disposed within the downstream flow path, the valve comprising:
a conical portion disposed adjacent the flow path hole; and
a spring, wherein one end of the spring is connected to the conical portion and the other end of the spring engages with the stop member.

18. The intraocular drainage device of claim 17, wherein the conical portion of the valve is configured to move downstream in response to an increased fluid pressure on the conical portion, thereby compressing the spring.

19. The intraocular drainage device of claim 17, wherein the valve is configured to allow an aqueous humour fluid to flow through the flow path hole, around an exterior surface of the conical portion, through a center portion of the spring and out the exit flow path.

20. The intraocular drainage device of claim 1, wherein the self-clearing device is a valve, the valve comprising:
a valve membrane; and
a flow path hole disposed in the center of the valve membrane, wherein the valve membrane is configured to bulge downstream in response to an increased fluid pressure on valve membrane, thereby increasing the size of the flow path hole.
